(19) United States
(12) Patent Application Publication
(10) Pub. No.: US 2005/0125025 A1
(43) Pub. Date: Jun. 9, 2005

(54) Styptic device

(76) Inventor: Marcel Rioux, Piedmont (CA)

Correspondence Address: Louis Tessier, 60 Balfour, T.M.R., QC, H3P 1L6, CA

(21) Appl. No.: 11/005,290

(22) Filed: Dec. 6, 2004

Related U.S. Application Data
(60) Provisional application No. 60/526,918, filed on Dec. 5, 2003.

(51) Int. Cl. A61B 17/00

(52) U.S. Cl. 606/201; 606/157

(57) ABSTRACT

A styptic device for hemostatically sealing a percutaneous vascular puncture formed in a body vessel and located at a target anatomical area of an intended patient includes a base component positionable substantially adjacent the target anatomical site. The device also includes a mounting component coupled to the base component for mounting the base component on the intended patient in a substantially adjacent relationship relative to the target anatomical site. The device further includes a compressing component defining a compressing surface for exerting a compressing pressure on the target anatomical site. The compressing component is operatively coupled to the base component so as to be movable relative to the latter between a compressing configuration wherein the compressing surface exerts the compressing force on the target anatomical site and a retracted configuration wherein the compressing surface is spaced from the target anatomical site.

This application claims the benefit of Provisional Patent Application Ser. No. 60/526.918 filed Dec. 5, 2003.

### FIELD OF THE INVENTION

The present invention relates to the general field of medical devices and is particularly concerned with a device for haemostatically sealing percutaneous vascular punctures.

### BACKGROUND OF THE INVENTION

There exists a plurality of medical and/or surgical procedures that are carried out intravascularly or intralumenally. For example, in the treatment of vascular diseases, such as atherosclerosis, angioplasty and/or stenting are now widely accepted procedures.

Such procedures usually involve the percutaneous puncture and insertion of a hollow needle through a patient's skin and muscle tissue into the vascular system. A guide wire is then typically passed through the needle lumen into the patient's blood vessel accessed by the needle. The needle may be removed, and an introducer sheath may be advanced over the guide wire into the vessel, for example, in conjunction with or subsequent to, a dilator.

A catheter or other device may then be advanced through a lumen of the introducer sheath and over the guide wire into position for performing a medical procedure such as, dilating the vessel, stenting of the latter, or the like.

In percutaneous transluminal coronary angioplasty, the catheter is typically introduced either in the radial or femoral artery and advanced through the artery to the coronary region. When the femoral artery is used, the latter may be located one half inch or more beneath the skin. Catheters typically have a diameter in the range of one millimetre and four millimetres, hence creating a significant puncture in the artery. Also, during the procedure, the catheter may be twisted or otherwise manipulated as it is advanced to the treatment site, hence potentially causing a further enlargement of the puncture.

Upon completion of the procedure, the devices and introducer sheath may be removed, leaving a puncture site in the vessel wall. Such procedures hence unavoidably present the problem of stopping the bleeding at the percutaneous puncture site after the procedure has been completed and after the instrument and any introducer sheaths used therewith have been removed.

At present, such bleeding is typically stopped by the application of direct digital pressure over the puncture site by a trained physician or other suitably trained medical personnel. Such direct pressure has to be properly applied for a sufficiently long period of time for haemostasis to occur so that the opening is effectively closed against further bleeding. The application of direct digital pressure over the puncture site, although somewhat useful, nevertheless suffers from numerous drawbacks.

First, the direct digital pressure application procedure constitutes an inefficient, if not wasteful, use of medical professional services. For example, in the case of punctures into relatively high pressure vessels, such as into the femoral artery or superficial femoral arteries, the pressure may have to be applied for as long as forty-five minutes for haemostasis to occur.

Second, the application of digital pressure over a relatively long period of time may result in fatigue, numbness, stiffness and/or pain occurring in the fingers, hands, wrists and/or forearms of the practitioner performing the procedure. Furthermore, repetition of the procedure over a period of time may cause repetitive-type stress injuries, such as carpal tunnel syndrome or the like.

Still furthermore, although the procedure is typically performed with gloves there exists a possibility that the glove could already have, or may develop, a tear, thereby allowing direct pressurized digital contact with potentially contaminating bodily fluids.

Third, it is often difficult for an individual to exert digital pressure of optimal magnitude, especially over a relatively long period of time. The magnitude of the pressure exerted may however prove to be particularly important in some situations. Indeed, should the magnitude of the pressure be suboptimal, a bruise or haematoma may form at the entry site since internal bleeding of the punctured artery continues until clotting blocks the puncture. On the contrary, should the applied pressure be too great, this may result in a substantial reduction, if not virtual arrest, of the flow of blood through the vessel. This, in turn, may lead to thrombosis of the vessel with potentially serious complications.

Yet another drawback associated with the conventional digital application of pressure at the puncture site results from the fact that the instrument and any introducer sheath used therewith is typically completely withdraw prior to the application of pressure at the puncture site. This results in a brief, yet vigorous, free-flow of blood through the puncture site, which may obscure the exact location of the puncture momentarily leading to further blood loss.

Still furthermore, the conventional method of digital pressure application of the puncture site is sometimes considered uncomfortable for the patient and requires that the patient remain immobilized in the operating room, catheter lab, holding area or the like hence using up valuable space.

Some styptic devices as been shown in the prior art, as exemplified by U.S. Pat. No. 5,307,811 issued May 3, 1994 to Ulrich Sigwart and Dan Akerfeldt. However, such prior art devices suffer from numerous drawbacks. Indeed, prior art device generally suffer from being unergonomical to the user and uncomfortable to the patient. Prior art devices also suffer from being overall too complex, and, hence, relatively expensive and potentially less reliable. Some prior art devices further suffer from requiring an external supporting surface and being deprived of a suitable pressure indicator.

Accordingly, there exists a need for an improved styptic device for hemostatically sealing percutaneous vascular punctures. It is a general objective of the present invention to provide such an improved styptic device.

### SUMMARY OF THE INVENTION

In accordance with an embodiment of the invention, there is provided a styptic device for at least substantially hemostatically sealing a percutaneous vascular puncture formed in a body vessel and located at a target anatomical area of an intended patient, the target anatomical area being surrounded by a surrounding area of the intended patient, the styptic device comprising a base component positionable substantially adjacent the target anatomical site; a mounting component coupled to the base component for mounting the base component on the intended patient in a substantially adjacent relationship relative to the target anatomical site; a compressing component defining a compressing surface for exerting a compressing pressure on the target anatomical site, the compressing component being operatively coupled to the base component so as to be movable relative to the latter between a compressing configuration wherein the compressing surface exerts the compressing force on the target anatomical site and a retracted configuration wherein the compressing surface is spaced from the target anatomical site.

Typically, the base component includes a base component socket section, the base component socket section defining a socket channel extending therethrough, the socket channel being positionable substantially over the target anatomical site; the compressing component extending at least partially through the socket channel for movement between the compressing and retracted configurations.

Conveniently, the compressing component includes a compressing component coupling section for coupling the compressing component to the base component, the compressing component coupling section having a substantially cylindrical configuration and being provided, at least in part, with a coupling component external thread; the socket channel being provided, at least in part, with a socket internal thread for threaded engagement with the coupling component external thread.

Typically, the compressing component defines a compressing component first longitudinal end and a longitudinally opposed compressing component second longitudinal end; the compressing component defining a compressing component grasping section located substantially adjacent the compressing component first longitudinal end for facilitating the manipulation of the compressing component and a compressing component contacting section for contacting the target anatomical site, the compressing component contacting section defining the compressing surface.

Conveniently, the compressing component grasping section includes a grasping knob extending substantially radially outwardly from the compressing component adjacent the compressing component first longitudinal end.

Conveniently, the compressing component contacting section includes a contacting tongue, the contacting tongue providing the compressing surface with a substantially rectangular configuration defining a contacting surface long axis and a substantially perpendicular contacting surface short axis, the contacting tongue being configured, sized and positioned so that the contacting surface long axis extends substantially in the direction of the body vessel when the compressing component is in the compressing configuration.

Typically, the base component also includes a base component spacing section for maintaining the base component socket section in a substantially overlying spaced relationship relative to the target anatomical site.

Typically, the mounting component includes a mounting component mounting plate extending from the base component for contacting at least part of the surrounding area, the mounting component mounting plate being provided with a mounting plate access aperture extending therethrough for allowing access to the target anatomical site; and wherein the base component spacing section includes a spacing section peripheral wall extending between the mounting component mounting plate and the base component socket section for maintaining the base component socket section in a spaced relationship relative to the mounting component mounting plate; the spacing section peripheral wall being provided with a peripheral wall access aperture extending therethrough for allowing access to the mounting plate access aperture.

Conveniently, the spacing section peripheral wall includes a peripheral wall first section extending substantially perpendicularly from the mounting component mounting plate and a substantially frusto-conical peripheral wall second section extending from the peripheral wall first section, the peripheral wall second section defining a finger abutment area for allowing abutting contact therewith of some of the fingers of the intended user.

Typically, the peripheral wall first and second sections are circumferentially truncated so as to define the peripheral wall access aperture, the peripheral wall access aperture defining a peripheral wall access aperture peripheral edge, the peripheral wall access aperture peripheral edge defining a pair of peripheral edge first segments formed in the peripheral wall first section and extending substantially perpendicularly from the mounting component mounting plate and a radially inwardly recessed peripheral edge second segment formed in the peripheral wall second section and extending between the peripheral edge first sections.

Typically, the mounting plate access aperture defines a mounting plate access aperture protruding portion projecting outwardly from the spacing section beyond the peripheral wall access aperture peripheral edge.

Preferably, the spacing section defines a barrier receiving chamber for receiving a pressure transmitting contamination barrier component, the barrier receiving chamber being configured, positioned and sized so that the pressure transmitting contamination barrier component is positioned intermediate the compressing surface and the target anatomical site when the compressing component is in the compressing configuration.

Conveniently, the compressing component coupling section defines a first threaded portion located substantially towards the compressing component first longitudinal end, a second threaded portion located substantially towards the compressing component second longitudinal end and an unthreaded portion located intermediate the first and second threaded portions; the dimensional relationship between the base component socket section, the compressing component and the base component spacing section being such that when the second threaded portion threadably engages the socket internal thread, the compressing component is in a stand-by configuration wherein the compressing component contacting section is located substantially adjacent the base component socket section above the level of the mounting component mounting plate so as to be in an overlying spaced apart relationship relative to the target anatomical site when the styptic device is mounted on the intended patient; when the lowermost thread of the second threaded portion threadably disengages the coupling component internal thread so as to be moved underneath the socket channel, the unthreaded portion is put in register with the socket channel allowing the compression component to drop under the action of gravity to a compressing component contacting configuration wherein the lowermost thread of the first threaded portion is put into contact with the uppermost thread of the socket channel and the compressing surface is positioned substantially in register with the mounting component mounting plate so as to contact the target anatomical site when the styptic device is mounted on the patient; when the first threaded portion threadably engages the coupling component internal thread, the compressing component is in the compressing configuration with the compressing surface located underneath the mounting component mounting plate so as to compress the target anatomical site when the styptic device is mounted on the patient.

Typically, the mounting component mounting plate defines a mounting plate outer peripheral edge, the mounting plate outer peripheral edge being provided with a groin accommodating recess formed therein for accommodating the groin region of the intended patient.

Conveniently, the mounting component also includes a mounting plate adhesive material attached to the mounting component mounting plate for adhesively securing the mounting plate to the surrounding area.

Typically, the mounting component also includes at least one strap extending from the mounting component mounting plate for at least partially surrounding a body part of the intended patient.

Conveniently, the mounting component includes a main strip section extending substantially laterally from the mounting component mounting plate and an auxiliary strip section extending from the mounting component mounting plate substantially opposite the main strip section the main and auxiliary strip sections being configured and sized for together encircling a body part of the patient and being provided with complementary main-to-auxiliary strap attachment means for releasable attachment threrbetween when together encircling the body part.

Typically, the mounting component includes a first auxiliary strip section and a second auxiliary strip section both extending substantially laterally from the mounting component mounting plate, the first and second auxiliary strips diverging outwardly away from each other; the mounting component also including a main strip section extending from the mounting component mounting plate substantially opposite the first and second auxiliary strip sections; the first and second auxiliary strip sections and the main strip section being configured, sized and oriented so that the main strip section is adapted to substantially encircle the body part and releasably attach to the first and second auxiliary strip sections substantially transversally relative to the latter.

Typically, the first and second auxiliary strip sections are configured, sized and oriented so as to extend substantially in register respectively with the external portion of the corresponding thigh and the buttocks region of the patient while the main strip section is adapted to extend from the corresponding groin region of the patient, substantially around the corresponding thigh of the patient to the first and second strip sections when the compressive surface is substantially in register with the femoral artery of the patient

Conveniently, either one of the main strip section or the first and second auxiliary strip sections is provided with a strip adhesive material attached thereto for adhesively securing the either one of the main strip section or the first and second auxiliary strip sections to the surrounding area.

Typically, the styptic device further comprises an alignment means for facilitating the alignment of the pressure contacting surface with the body vessel.

Conveniently, the alignment indicia includes a first alignment indicia located on the compressing component contacting section and a second alignment indicia located on the base component, the first and second alignment indicia extending in a substantially perpendicular relationship relative to each other and being located so that the intersection of the projection thereof defines an alignment intersection adapted to be put in register with the target anatomical area of the patient.

Preferably, the styptic devise further comprises a pressure sensor for sensing the styptic pressure exerted by the compressing component on the target anatomical site and providing a usable indication of the magnitude of the styptic pressure.

Preferably, the pressure sensor provides an electrical output, the electrical output being transmittable through a corresponding sensor cable; whereby the sensor cable is adapted to be coupled to a pressure indicator.

In accordance with an alternative embodiment of the invention, the pressure sensor includes a compressible bladder mounted on the compressing surface, the compressible bladder being fluidly coupled to a magnitude indicating tube, the compressible bladder being filled, at least in part, by a substantially non-compressible fluid, the magnitude indicating tube being provided with a buoyant magnitude indicating component adapted to float on top of the non-compressible fluid and to move longitudinally within the magnitude indicating tube; whereby upon the styptic pressure being applied to the target anatomical site, the compressible bladder will be squeezed between the compressing surface and the target anatomical site, upon being squeezed, the compressible bladder will be compressed, forcing the non-compressible fluid into the magnitude indicating tube and modifying the level of the magnitude indicating component within the magnitude indicating tube.

In accordance with yet another embodiment of the invention, the pressure sensor includes a pressure rod mechanically coupled to the compressing surface, the pressure rod being slidably mounted within the compressing component for longitudinal movement threrein towards the compressing component first longitudinal end upon a pressure being exerted on the compressing surface; a biasing means coupled to the pressure rod for biasing the compressing component second longitudinal end; a pressure indicator mounted on the compressing component; a converting means coupled to the pressure indicator and to the pressure rod for converting the movement of the pressure rod into a pressure data indicated by the pressure indicator; whereby the biasing means is calibrated so that the pressure data corresponds substantially to the magnitude of pressure exerted by the compressing surface on the target anatomical area.

Advantages of the present invention include that the proposed device allows for haemostatic sealing of vascular puncture sites through a set of quick and ergonomic steps, without requiring special tooling or manual dexterity.

Furthermore, the proposed method and device allows for haemostatic sealing without requiring the direct intervention through the application of direct digital pressure for extended periods of time. Consequently, the proposed method and device allows physicians, nurses or other trained medical personnel to be used more efficiently.

The proposed method and device also allows other medical resources to also be used more efficiently since it allows the patient to be removed from the operating room, catheter lab, holding area or other valuable space once the device has been properly installed over the puncture site.

By eliminating the need for digital application of pressure over relatively long periods of time, the proposed method and device also reduces the occurrence of both acute and repetitive-type injuries to medical practitioners associated with the conventional method. The risk of contamination with bodily fluids is also substantially reduced.

Still furthermore, the proposed method and device allows for calibration of the pressure exerted at the puncture site and further allows the pressure to remain at the calibrated magnitude over the required period of time. Accordingly, the proposed method and device reduces the risks of haematoma, thrombosis or other problems associated with the application of pressures of improper magnitude.

Another advantage associated with the present invention resides in that the proposed device is designed so as to be comfortable for the patient during installation, use and removal thereof.

Yet still furthermore, the proposed device is designed so as to be easily scalable and/or configurable so as to allow customization thereof to various types of situations such as to various puncture sites and/or for various types of medical and/or surgical procedures.

Yet still furthermore, the proposed device is designed so as to be manufacturable using conventional materials and conventional forms of manufacturing so as to provide a device that will economical to manufacture. The device is also designed so as to be reliable and relatively trouble-free in operation.

### BRIEF DESCRIPTION OF THE DRAWINGS

Various embodiments of the present invention will now be disclosed, by way of example, in reference to the following drawings, in which:

FIG. 1, in a partial top view with sections taken out, illustrates the styptic device in accordance with an embodiment of the present invention;

FIG. 2, in a perspective view, illustrates some of the components part of the styptic device shown in FIG. 1;

FIG. 3, in a top view, illustrates the components shown in FIG. 2;

FIG. 4, in a right-hand side view, illustrates the components shown in FIGS. 2 and 3;

FIG. 5, in a front view, illustrates the components shown in FIGS. 2 through 4;

FIG. 6, in a left-hand view, illustrates the components shown in FIGS. 2 through 5;

FIG. 7, in a rear view, illustrates the components shown in FIGS. 3 through 6;

FIG. 8, in a bottom view, illustrates the components shown in FIGS. 2 through 7;

FIG. 9, in a partially exploded view, illustrates the components shown in FIGS. 2 through 8;

FIG. 10, in a partial longitudinal cross-sectional view with sections taken out, illustrates some of the features of a base component part of the styptic device shown in FIGS. 1 through 9;

FIG. 11, in a partial close-up view, illustrates some of the features of the components shown in FIG. 10;

FIG. 12, in a top view, illustrates a grasping knob part of the styptic device shown in FIGS. 1 through 11;

FIG. 13, in a partial exploded view with sections taken out, illustrates some of the features of components part of the styptic device shown in FIGS. 1 through 12;

FIG. 14, in a transversal cross-sectional view, illustrates the configuration of the grasping knob shown in FIG. 12;

FIG. 15, in a partial detailed view taken inside circle D of FIG. 13, illustrates some of the features of the grasping knob shown in FIGS. 12 through 14;

FIG. 16, in a longitudinal cross-sectional view, illustrates some of the components of the styptic device shown in FIGS. 1 through 15, the compressing component thereof being shown in a stand-by configuration;

FIG. 17, in a longitudinal cross-sectional view, illustrates the components shown in FIG. 16, with the compressing component shown in a contacting configuration;

FIG. 18, illustrates the components shown in FIGS. 16 and 17 with the compressing component shown in a compressing configuration;

FIG. 19, in a bottom view, illustrates the styptic device shown in FIG. 1;

FIG. 20, in an exploded view, illustrates some of the sections of a mounting component part of a styptic device in accordance with an embodiment of the present invention;

FIG. 21, in a perspective view, illustrates part of a styptic device in accordance with an alternative embodiment of the invention wherein the styptic device is provided with an alternative pressure sensing assembly;

FIG. 22, in a partial bottom view with sections taken out, illustrates some of the features of the embodiment shown in FIG. 21;

FIG. 23, in a partial longitudinal cross-sectional view, illustrates some of the features of the embodiment shown in FIGS. 21 and 22;

FIG. 24, in a perspective view, illustrates part of a styptic device in accordance with yet another alternative embodiment of the invention wherein the styptic device is provided with an alternative pressure sensing assembly;

FIG. 25, in a bottom view, illustrates one of the initial steps for mounting a styptic device in accordance with an embodiment of the present invention to the body of an intended patient, FIG. 25 showing an intended user removing a peelable covering strip from an adhesive tape mounted on a mounting plate part of a styptic device in accordance with an embodiment of the present invention;

FIG. 26, in a partial top view, illustrates the mounting plate being adhesively secured to the body of the intended patient;

FIG. 27, illustrates a strap assembly being wrapped around the thigh of an intended patient;

FIG. 28, illustrates a styptic device in accordance with an embodiment of the invention mounted on an intended patient and in a stand-by configuration;

FIG. 29, illustrates a sterile gauze being inserted between a contacting tongue part of a styptic device in accordance with an embodiment of the present invention and a target anatomical area of the intended patient;

FIG. 30, illustrates the hand of an intended user grasping a grasping knob part of a compressing component part of the styptic device for moving the compressing component towards its compressing configuration;

FIG. 31, illustrates an intended user exerting pressure on the target anatomical site as a catheter is being removed therefrom; and

FIG. 32, illustrates the styptic device mounted on the intended patient with its compressing component in a compressing configuration.

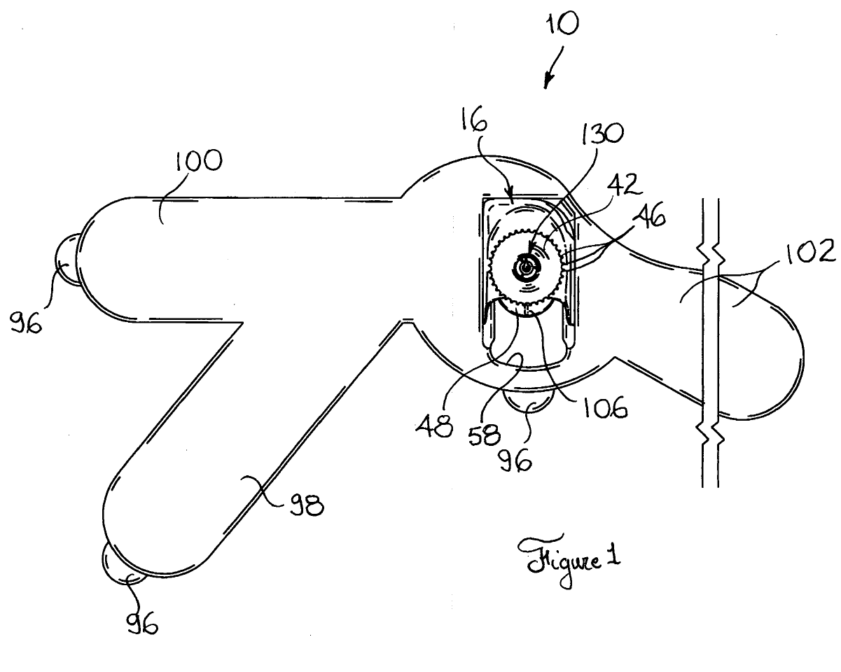

### DETAILED DESCRIPTION

Referring to FIG. 27, there is shown a styptic device in accordance with an embodiment of the present invention, generally indicated by the reference numeral 10. The styptic device 10 is intended to be used mainly for haemostatically sealing percutaneous vascular punctures. It should, however, be understood that the device 10 could be used in numerous other contexts as a guiding means, and/or pressure creating means, for respectively guiding the insertion of a medical or surgical tool during insertion thereof in a body tissue and/or exerting a predetermined pressure on a body site, without departing from the scope of the present invention.

Also, the styptic device 10 in accordance with the present invention is hereinafter disclosed mainly for use in the specific context of the cannulation of the femoral artery for the purpose of heart catheterization or coronary angioplasty. It should, however, be understood that the styptic device 10 could be designed for other types of cannulation at other body sites without departing from the scope of the present invention. For example, the present invention could easily be scaled and configured for cannulation at radial body sites and/or for drawing arterial blood gases, or any other suitable purpose.

The styptic device 10 is typically used for at least substantially haemostatically sealing a percutaneous vascular puncture formed in a body vessel (not shown) and located at a target anatomical area 12 of an intended patient. The target anatomical area 12 is surrounded by a surrounding area 14 of the intended patient.

In general terms, the styptic device 10 includes a base component 16 positionable substantially adjacent the target anatomical site 12. The styptic device 10 also includes a mounting component 18 coupled to the base component 16 for mounting the base component 16 on the intended patient in a substantially adjacent relationship relative to the target anatomical site 12.

The styptic device 10 further includes a compressing component 20 defining a compressing surface 22 for exerting a compressing pressure on the target anatomical site 12. The compressing component 20 is operatively coupled to the base component 16 so as to be moveable relative to the latter between a compressing configuration and a retracted configuration. In the compressing configuration shown, for example, in FIG. 31, the compressing surface 22 exerts the compressing force on the target anatomical site 12. In the retracted configuration, shown for example in FIG. 28, the compressing surface 22 is spaced from the target anatomical site 12.

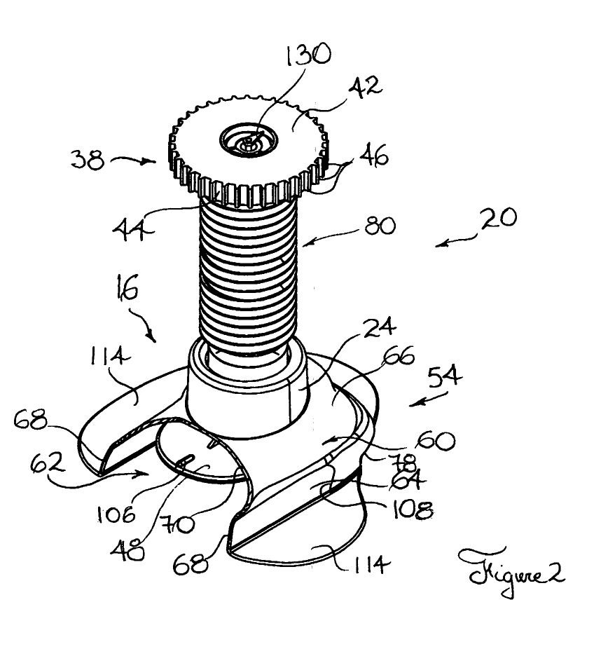

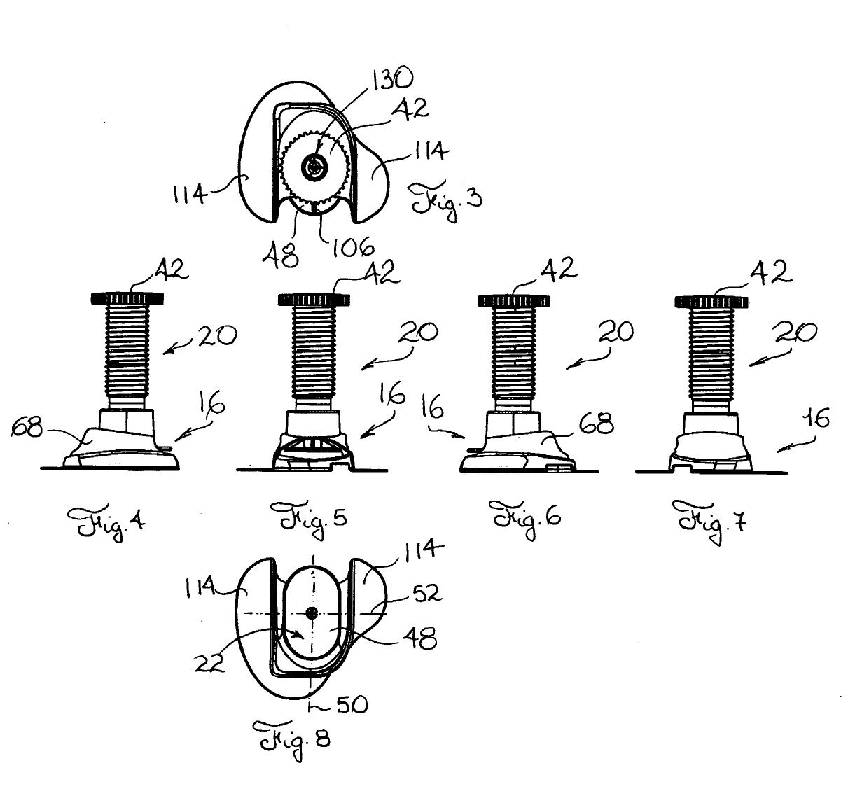

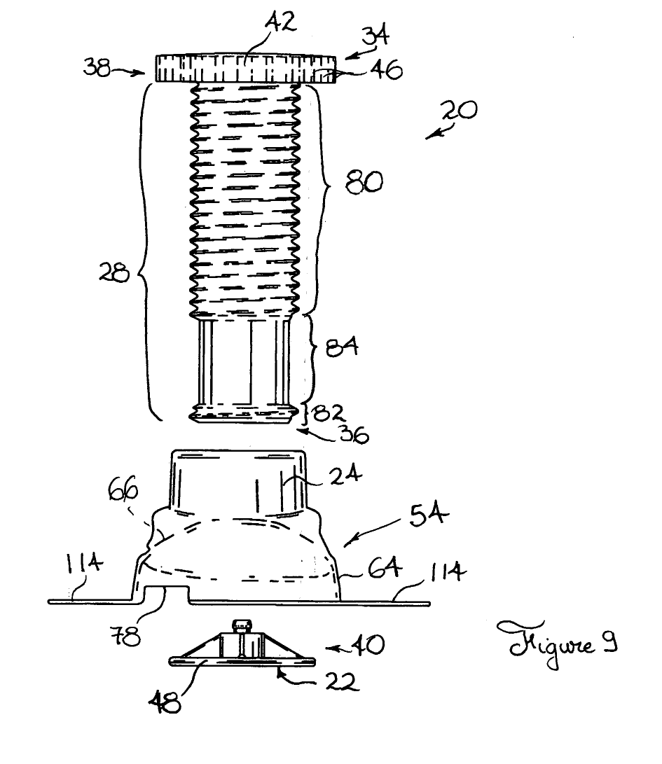

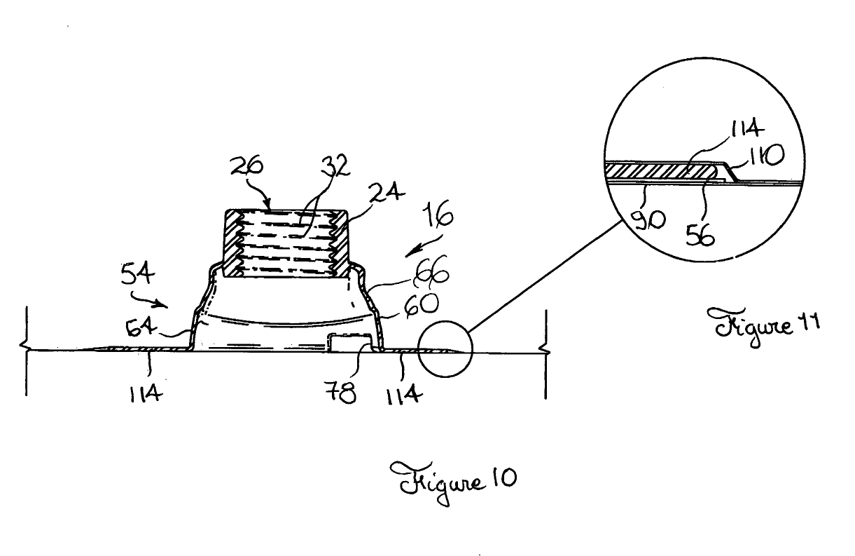

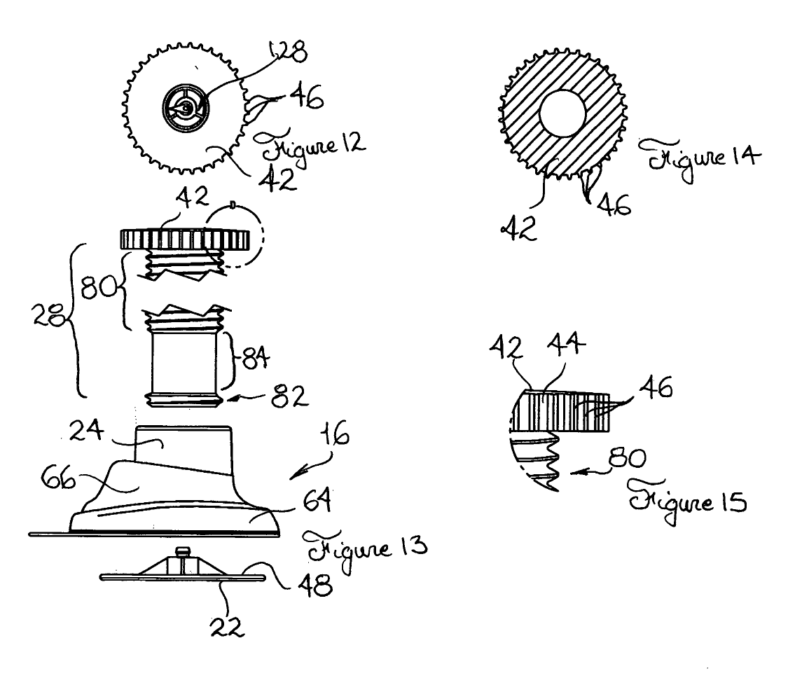

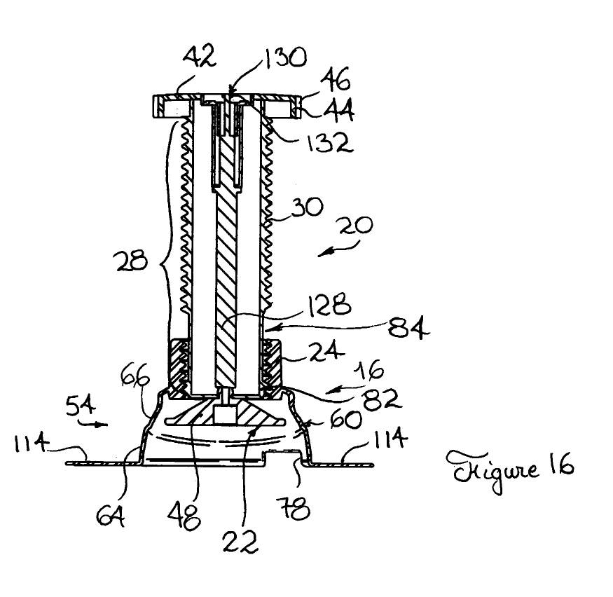

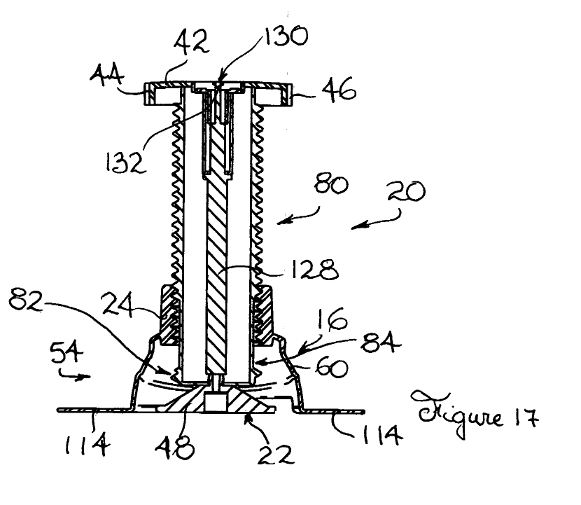

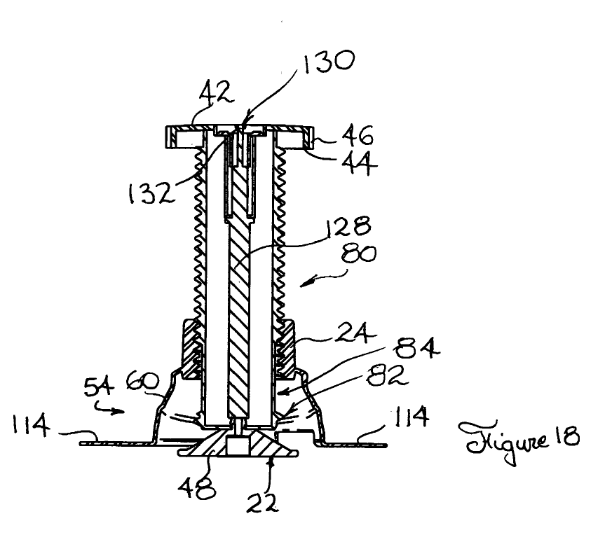

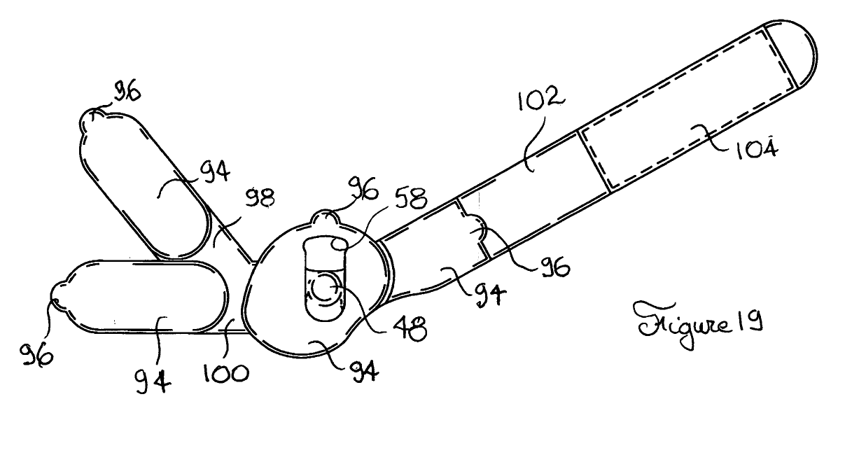

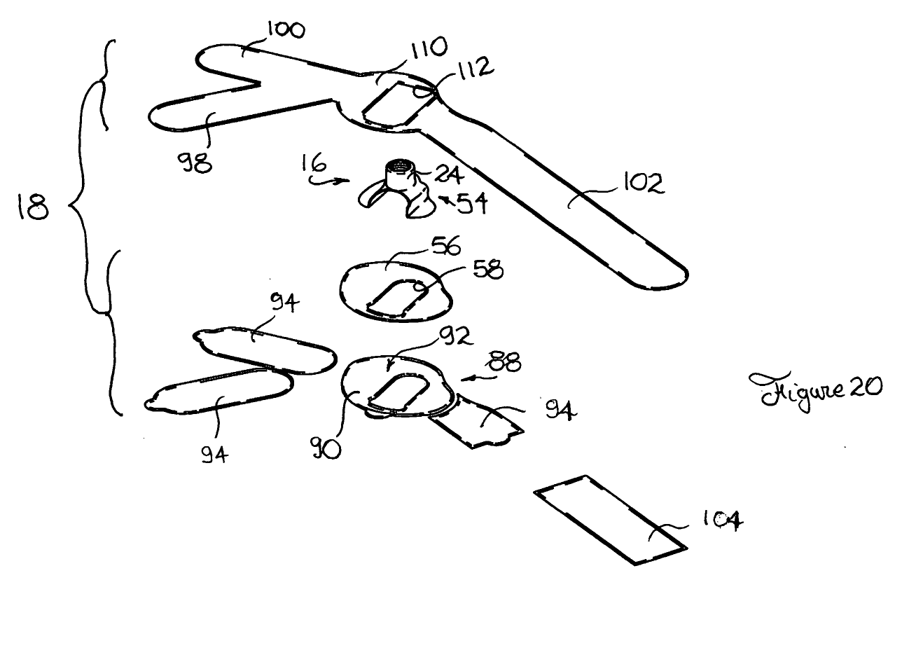

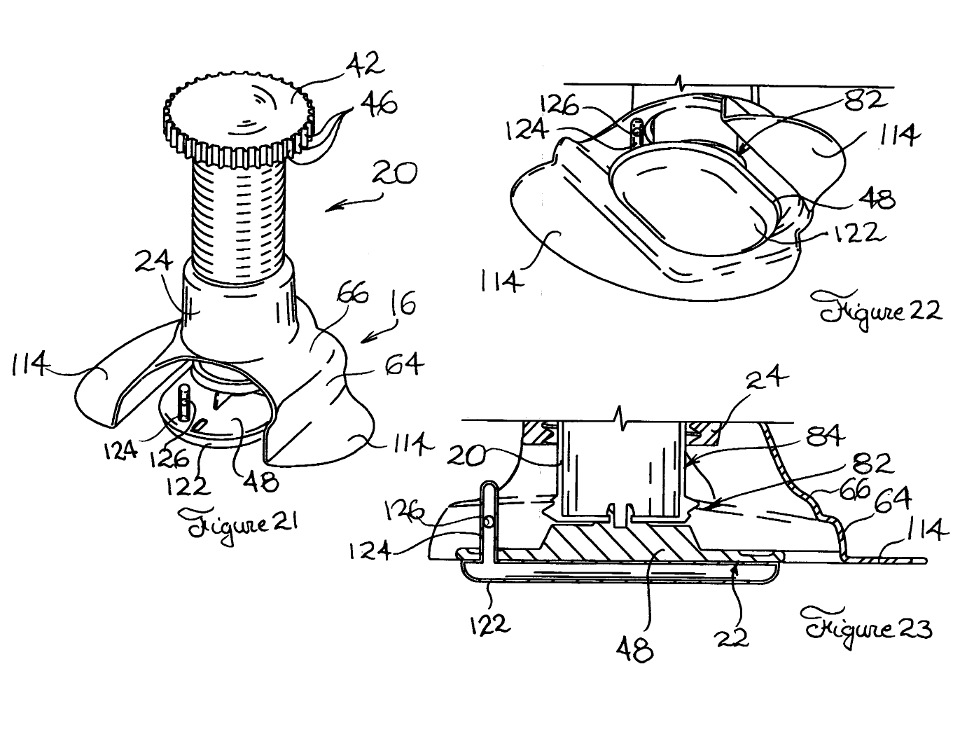

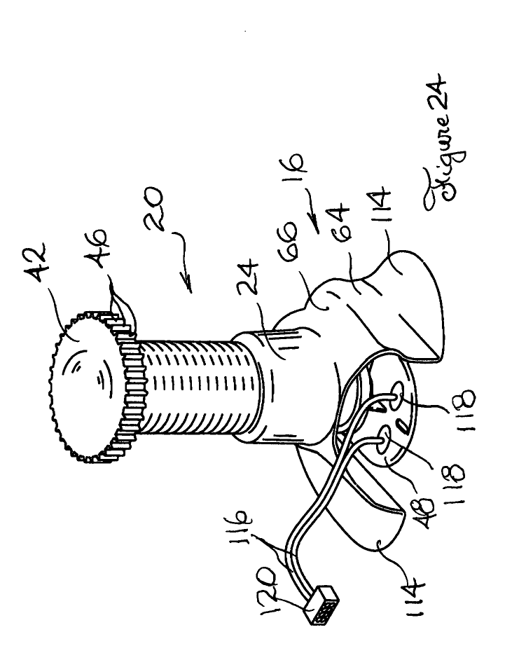

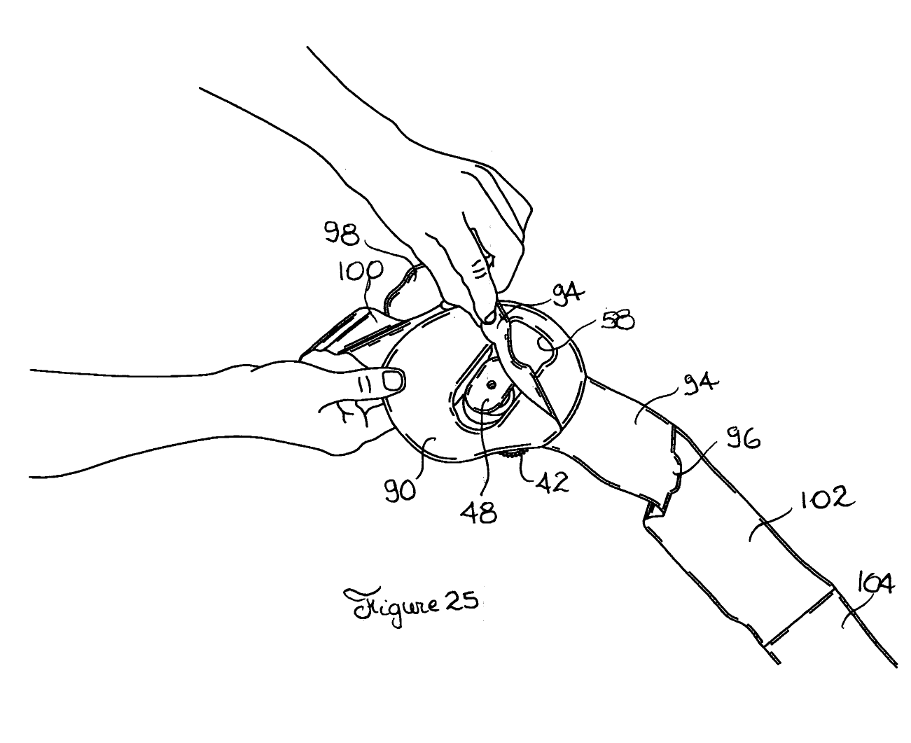

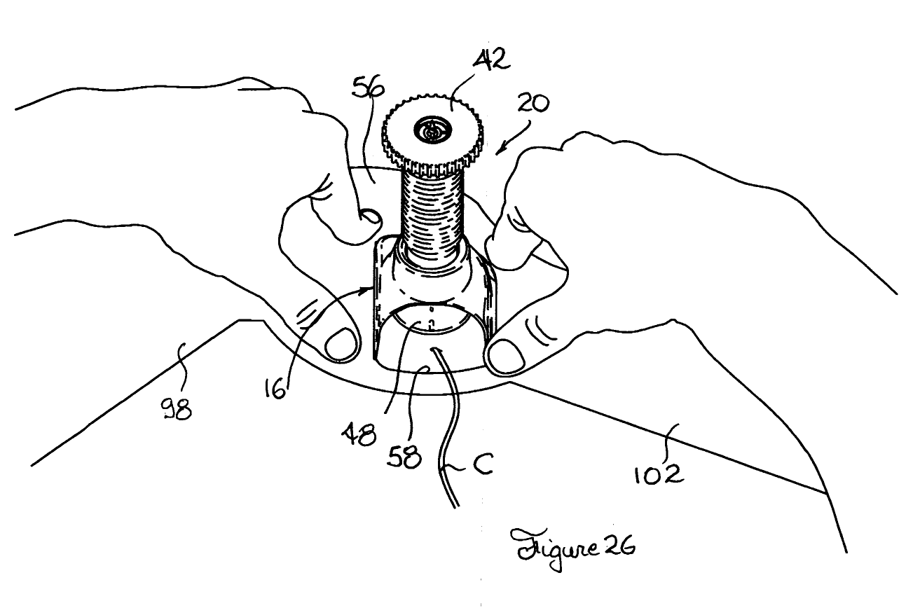

Referring now more specifically to FIGS. 2 through 18, there is shown some of the features of the base and compressing components 16, 20. As shown more specifically in FIGS. 10 and 16 through 18, the base component 16 includes a base component socket section 24. The base component socket section 24 defines a socket channel 26 extending longitudinally therethrough. The socket channel 26 is typically positionable substantially over the target anatomical site 12.

The compressing component 20 extends at least partially through the socket channel 26 for movement between the compressing and retracted configurations. The compressing component 20 includes a compressing component coupling section 28 for coupling the compressing component 20 to the base component 16.

The compressing component coupling section 28 typically has a substantially cylindrical configuration and is provided, at least in part, with a coupling component external thread 30. The socket channel 26 is provided, at least in part, with a complementary socket internal thread 32 for threaded engagement with the coupling component external thread 30.

As illustrated more specifically in FIG. 9, the compressing component 20 defines a compressing component first longitudinal end 34 and a longitudinally opposed compressing component second longitudinal end 36. The compressing component 20 defines a compressing component grasping section 38 located substantially adjacent the compressing component first longitudinal end 34 for facilitating the manipulation of the compressing component 20.

The compressing component 20 also defines a compressing component contacting section 40 for contacting the target anatomical site 12. The compressing component contacting section 40 defines the compressing surface 22.

Typically, the compressing component grasping section 38 includes a grasping knob 42 extending substantially radially outwardly from the compressing component 20 adjacent the compressing component first longitudinal end 34. Typically, although by no means exclusively, the grasping knob 42 has a substantially disc-shaped configuration. As shown more specifically in FIGS. 16 through 18, the grasping knob 42 typically includes a knob flange 44 depending therefrom. The knob flange 44 is typically provided with friction enhancing protrusions 46 extending radially outwardly therefrom.

As shown in FIG. 9, the compressing component contacting section 40 typically includes a contacting tongue 48. As shown more specifically in FIG. 8, the contacting tongue 48 typically provides the compressing surface 22 with a substantially rectangular or ovaloïd configuration defining a contacting surface long axis 50 and a substantially perpendicular contacting surface short axis 52. The contacting tongue 48 is configured, sized and positioned so that the contacting surface long axis 50 extends substantially in the direction of the body vessel when the compressing component 20 is in the compressing configuration.

As shown throughout the Figures, the contacting tongue 48 is typically releasably attached to the compressing component coupling section 28. It should, however, be understood that the contacting tongue 48 could be permanently fixed to the compressing component coupling section 28 without departing from the scope of the present invention or that other types of releasable coupling means could be used for releasably coupling the contacting tongue 48 to the compressing component coupling section 28 without departing from the scope of the present invention.

As illustrated more specifically in FIGS. 2, 10, and 16 through 18, the base component 16 also includes a base component spacing section 54 for maintaining the base component socket section 24 in a substantially overlying space relationship relative to the target anatomical site 12.

As illustrated more specifically in FIG. 20, the mounting component 18 typically includes a mounting component mounting plate 56 extending from the base component 16 for contacting at least part of the surrounding area 14. The mounting component mounting plate 56 is provided with a mounting plate access aperture 58 extending therethrough for allowing access to the target anatomical site 12 when the styptic device 10 is mounted on the intended patient.

As illustrated more specifically in FIGS. 2, 10, and 16 through 18, the base component spacing section 54 includes a spacing section peripheral wall 60 adapted to extend between the mounting component mounting plate 56 and the base component socket section 24 for maintaining the base component socket section 24 in a spaced relationship relative to the mounting component mounting plate 56. The spacing section peripheral wall 60 is provided with a peripheral wall access aperture 62 extending therethrough for allowing access to the mounting plate access aperture 58 when the styptic device 10 is mounted on the intended patient.

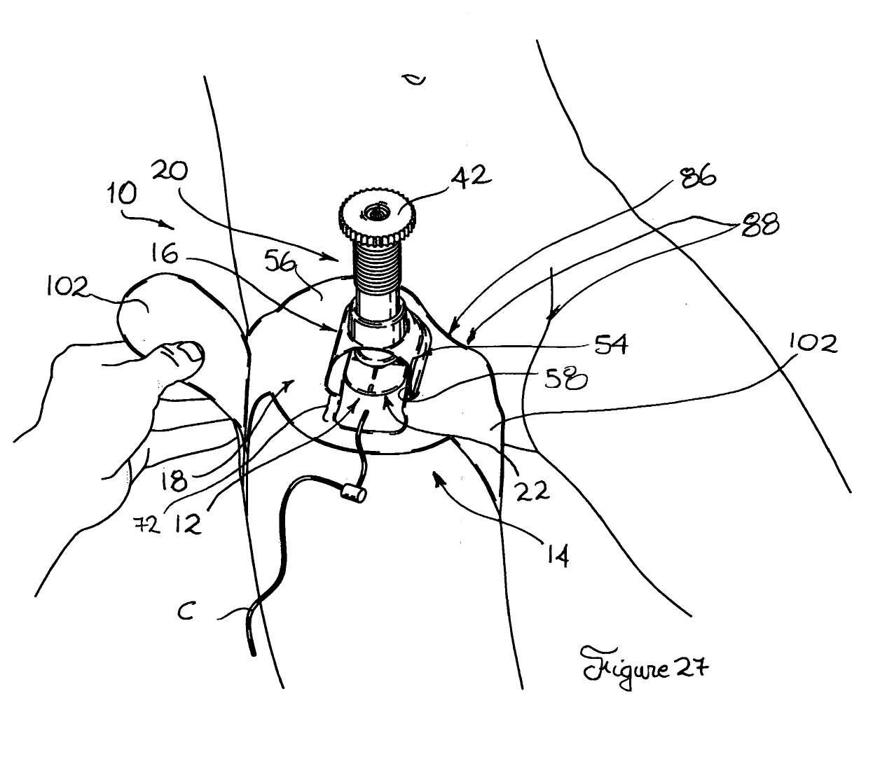

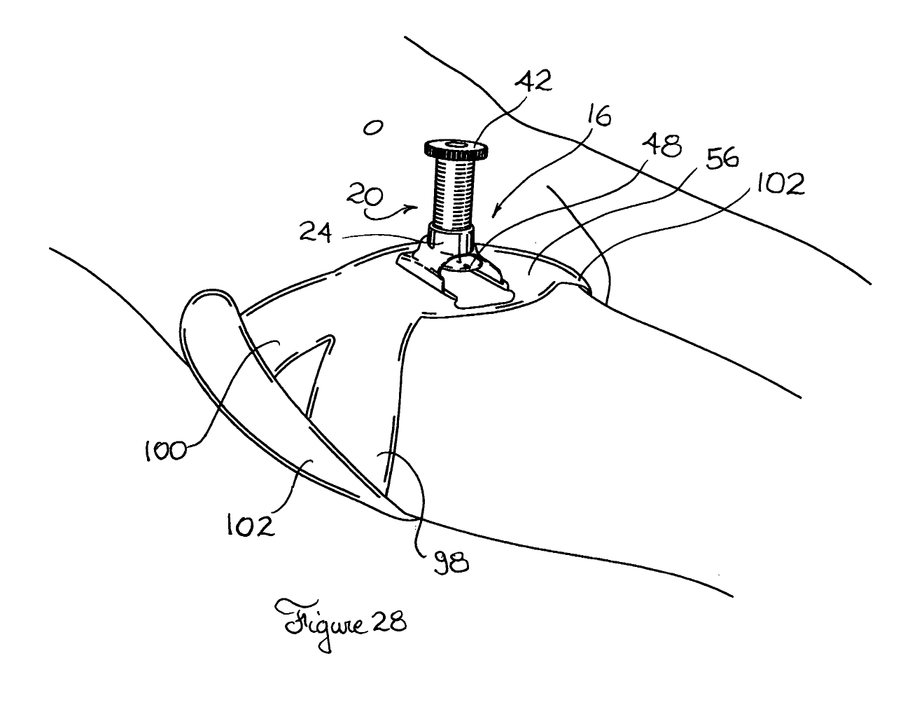

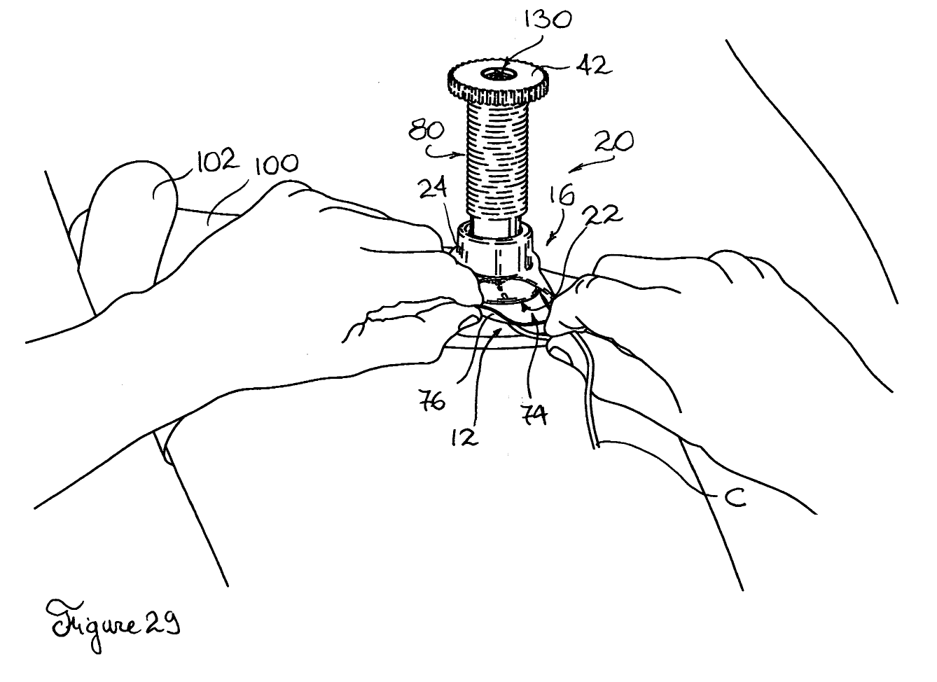

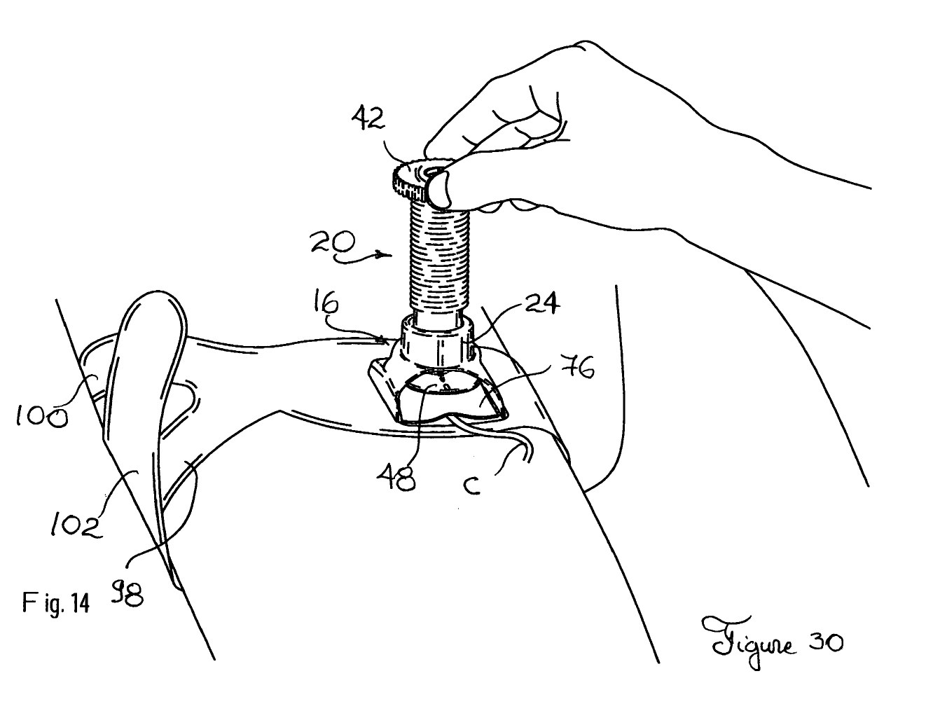

As illustrated more specifically in FIG. 2, the spacing section peripheral wall 60 includes a peripheral first section 64 adapted to extend substantially perpendicularly from the mounting component mounting plate 56 and a substantially frustro-conical peripheral wall second section 66 extending from the peripheral wall first section 64. As shown more specifically in FIGS. 4 and 6, the peripheral wall second section 66 defines a finger abutment area 68 for allowing abutting contact therewith of some of the fingers of the intended user.

Referring back to FIG. 2, there is shown that the peripheral wall first and second sections 64, 66 are circumferentially truncated so as to define the peripheral wall access aperture 62. The peripheral wall access aperture 62, in turn, defines a peripheral wall access aperture peripheral edge. The peripheral wall access aperture peripheral edge, in turn, defines a pair of peripheral edge first segments 68 formed in the peripheral wall first section 64 and extending substantially perpendicularly from the mounting component mounting plate 56.

The peripheral wall access aperture peripheral edge also defines a radially inwardly recessed peripheral edge second segment 70 formed in the peripheral wall second section 66 and extending between the peripheral edge first sections 68. The recessed peripheral edge second segment 70 is adapted to facilitate manipulation and visualization during operational steps associated with the use of the styptic device 10.

As illustrated more specifically in FIGS. 27, and 30, the mounting plate access aperture 58 defines a mounting plate access aperture protruding portion 72 projecting outwardly from the spacing section 54 beyond the peripheral wall access aperture peripheral edge. The mounting plate access aperture protruding portion 72 is also adapted to facilitate manipulation and visualization during operational steps associated with the use of the styptic device 10.

As illustrated more specifically in FIG. 28, the spacing section 54 defines a barrier-receiving chamber 74 for receiving a pressure transmitting contamination barrier component 76. The barrier receiving chamber 74 is configured, positioned and sized so that the pressure transmitting contamination barrier component 76 is positioned intermediate the compressing surface 22 and the target anatomical site 12 when the compressing component 20 is in the compressing configuration.

Typically, the pressure transmitting contamination barrier component 76 takes the form of a strip of material suitable for transmitting the compressive force exerted by the compressing component 20 on the target anatomical site 12 while acting as a biological barrier, or retardant, against contamination thereof by pathogenic organisms. Typically, although by no means exclusively, the pressure transmitting contamination barrier component may take the form of a sterile gauze or film or any other suitable medium. Typically, although by no means exclusively, the barrier receiving chamber is configured and sized for receiving a conventional 2-inch by 3-inch sterile gauze.

As illustrated more specifically in FIGS. 10, and 16 through 18, the peripheral wall first section 64 is typically provided with a barrier component receiving aperture 78 extending therethrough for allowing at least a portion of the contamination barrier component 76 to extend therethrough. Typically, the barrier component receiving aperture 78 allows a corner portion of the contamination barrier component 76 to extend therethrough so as to optimize the configuration of the barrier receiving chamber 74, taking into consideration other criteria such as the optimal shape of the mounting component mounting plate 56.

As illustrated more specifically in FIGS. 9, and 16 through 18, the compressing component coupling section 28 defines a first threaded portion 80 located substantially towards the compressing component first longitudinal end 34. The compressing component coupling section 28 also defines a second threaded portion 82 located substantially towards the compressing component second longitudinal end 36. The compressing component coupling section 28 further defines an unthreaded portion 84 located intermediate the first and second threaded portions 80, 82.

As shown more specifically in FIG. 16, the dimensional relationship between the base component socket section 24, the compressing component 20 and the base component spacing section 54 is such that when the second threaded portion 82 threadably engages the socket internal thread 32, the compressing component 20 is in a stand-by configuration. In the stand-by configuration, the compressing component contacting section 40 is located substantially adjacent the base component socket section 24 above the level of the mounting component mounting plate 56 so as to be in an overlying spaced apart relationship relative to the target anatomical site 12 when the styptic device 10 is mounted on the intended patient.

Typically, although by no means exclusively, the second threaded portion 82 includes a small number of threads such as a single thread. When the lowermost or only thread of the second threaded portion 82 threadably disengages the coupling component internal thread 32 so as to be moved underneath the socket channel 24, the unthreaded portion 84 is put in register with the socket channel 24 allowing the compression component 20 to drop under the action of gravity to a compressing component contacting configuration shown in FIG. 17. In the compressing component contacting configuration, the lowermost thread of the first threaded portion 80 is put in contact with the uppermost thread of the socket channel 24 and the compressing surface 22 is positioned substantially in register with the mounting component mounting plate 56 so as to contact the target anatomical site 12 when the styptic device 10 is mounted on the patient.

The dimensional relationship between the base component socket section 24, the compressing component 20 and the base component spacing section 54 is also such that when the first threaded portion 80 threadably engages the socket internal thread 32, the compressing component 20 is in the compressing configuration with the compressing surface 22 located underneath the mounting component mounting plate 56 so as to compress the target anatomical site when the styptic device 10 is mounted on the intended patient.

The mounting component mounting plate 56 defines a mounting plate outer peripheral edge. As shown more specifically in FIG. 27, the mounting plate outer peripheral edge is typically provided with a groin accommodating recess 86 formed therein. The groin accommodating recess 86 is configured and sized for accommodating a corresponding groin region 88 of the intended patient so as to improve the comfort of the latter.

As shown more specifically in FIG. 20, the mounting component 18 typically further includes a mounting plate adhesive material 88 adapted to be attached to the mounting component mounting plate 56 for adhesively securing the mounting component mounting plate 56 to the surrounding area 14. Typically, the mounting component adhesive material 88 includes a double-sided adhesive tape 90.

The double-sided adhesive tape 90 has a first side 92 thereof adhesively secured to the undersurface of the mounting component mounting plate 56 and a second side thereof (not shown) releasably covered by a peelable protective strip 94 (shown in FIG. 19). The peelable protective strip 94 typically has a strip prehension tongue 96 projecting outwardly therefrom to facilitate grasping of the peelable protective strip during the peeling thereof from the second side of the double-sided adhesive tape 90.

The double-sided adhesive tape 90 is typically configured and sized so as to substantially conform to the configuration of the mounting component mounting plate 56. Also, the double-sided adhesive tape 90 is typically provided with a mounting plate tape aperture 91 extending therethrough for facilitating manipulation and visualization during installation of the styptic device 10 on the intended patient.

Typically, the mounting component 18 also includes at least one strap extending from the mounting component mounting plate 56 for at least partially surrounding a body part of the intended patient. Typically, the mounting component 18 includes a first auxiliary strip section 98 and a second auxiliary strip section 100 both extending substantially laterally from the mounting component mounting plate 56. The first and second auxiliary strips 98, 100 typically diverge substantially outwardly away from each other.

The mounting component 18 also typically also includes a main strip section 102 extending from the mounting component mounting plate 56 substantially opposite the first and second auxiliary strip sections 98, 100. The first and second auxiliary strip sections 98, 100 and the main strip section 102 are configured, sized and oriented relative to the mounting component mounting plate 56 so that the main strip section 102 is adapted to substantially encircle the intended body part of the intended patient and releasably attached to the first and second auxiliary strip section 98, 100 substantially transversally relative to the latter.

In situations wherein the styptic device 10 is intended to be used for haemostatically sealing a percutaneous vascular puncture formed in the femoral artery of a patient, the first and auxiliary strip sections 98, 100 are configured, sized and oriented relative to the mounting component mounting plate 56 so as to be located respectively substantially in register with the exterior face of the thigh of the intended patient and at least a portion of the buttocks of the intended patient. In such situations, the main strip section 102 is configured and sized so as to encircle the thigh of the intended patient substantially from the groin area of the latter to the first and second auxiliary strips sections 98, 100.

It should, however, be understood that the configuration and size of the mounting component strap or straps may differ substantially from that shown throughout the drawings without departing from the scope of the present invention.

Typically, the main strip section 102 and the first and second auxiliary strip sections 98, 100 are adapted to be releasably attached together using suitable releasable attachment means. In a preferred embodiment of the invention, the main strip section 102 and the first and second auxiliary strip sections 98, 100 are provided with portions of complementary miniature hook and loop fibre located adjacent distal ends thereof for allowing the main strip section 102 to releasably attach to the first and second auxiliary strip sections 98, 100. As shown in FIGS. 19 and 20, a removable protective strip 104 is typically releasably attached to the portion of complementary miniature hook and loop fibre of the main strip section 102 for protection against soiling or contamination prior to use.

The mounting component 18 is typically further provided with strap adhesive material attached to the main strip section 102 and the first and second auxiliary strip sections 98, 100 for adhesively securing the latter to the surrounding area. Typically, the strap adhesive material includes corresponding double-sided adhesive tapes, each having a first side thereof adhesively secured to the corresponding strip section and a second side thereof releasably covered by a peelable protective strip 94.

The styptic device 10 typically further includes an alignment means for facilitating the alignment of the pressure contacting surface 22 with the body vessel (not shown). As shown more specifically in FIG. 2, the alignment means typically includes a first alignment indicia 106 located on the compressing component contacting section and a second alignment indicia 108 located on the base component 16. The first and second alignment indicia 106, 108 typically extend in a substantially perpendicular relationship relative to each other and are located so that the intersection of the projection thereof defines an alignment intersection location adapted to be put in register with the target anatomical site 12 of the intended patient.

Typically, although by no means exclusively, the first alignment indicia 106 includes an indicia protrusion or recess formed on the top surface of the contacting tongue 48 while the second alignment indicia 108 is grooved, traced or otherwise marked on the peripheral wall first section 64. It should, however, be understood that other types of alignment indicia could be used without departing from the scope of the present invention.

As illustrated more specifically in FIG. 20, the main strip section 102 and the first and second auxiliary strip sections 98, 100 typically merge integrally into a strip connection segment 110. The strip connecting segment 110 is secured to the mounting component mounting plate 56.

Typically, the strip connecting segment 110 is secured to the upper surface of the mounting component mounting plate 56. Also, typically, the strip connecting segment 110 is configured and sized so as to substantially conform to the configuration of the mounting component mounting plate 56. Hence, typically, the strip connecting segment 110 is typically provided with a connecting segment aperture 112 extending therethrough and adapted to be put in register with the mounting plate access aperture 58.

As shown throughout the Figures and, more specifically in FIG. 10, the base component 16 is provided with at least one and typically two base component attachment flanges 114 extending substantially laterally from the lower peripheral edge of corresponding peripheral wall first sections 64. As shown more specifically in FIG. 11, the base component attachment flanges 114 are typically secured between the mounting component mounting plate 56 and the strip connecting segment 110 for securing the base component 16 to the mounting component mounting plate 56.

The styptic device 10 is typically further provided with a pressure evaluating component or pressure sensor for allowing an intended user to evaluate the pressure exerted by the pressure component 20 on the target anatomical site.

In a preferred embodiment of the invention illustrated in FIG. 24, the pressure sensor provides an electrical output. The electrical output is transmittable through at least one corresponding sensor cable 116. The sensor cable 116 is adapted to be coupled to a suitable monitoring emitter.

Typically, although by no means exclusively, the pressure sensor may include piezo-electric components or any other suitable pressure sensing component 118. The pressure sensor is typically located on the contacting tongue 48 so as to be in contact with the compressing surface 22.

It should be understood that any suitable type of pressure sensor could be used without departing from the scope of the present invention. Also, the pressure sensor could be located at any suitable location without departing from the scope of the present invention. Furthermore, any suitable number of sensor cables 116 could be used with any suitable type of cable-to-emitter display plug 120 without departing from the scope of the present invention.

Furthermore, the monitoring emitted by the pressure indicator may be addressed to any sensorial modality. For example, the monitoring emitter may provide a visual display and/or an audio signal and/or a tactile signal without departing from the scope of the present invention.

Still furthermore, the monitoring emitter may provide a continuous indication as to the magnitude of the styptic pressure. It may also provide an alert signal upon the styptic pressure reaching a predetermined threshold or otherwise provide any other type of useful indication about the magnitude of the styptic pressure without departing from the scope of the present invention.

FIGS. 21 through 23 illustrate an alternative embodiment of a pressure sensor wherein a compressible bladder 122 is mounted on the compressing surface 22. The compressible bladder 122 is fluidly coupled to a magnitude indicating tube 124. Typically, although by no means exclusively, the compressible bladder 122 is mounted on the bottom surface of the contacting tongue 48 while the magnitude indicating tube 124 extends through the latter.

The compressible bladder 122 is filled, at least in part, by a substantially non-compressible fluid. The magnitude indicating tube 124 is provided with a buoyant magnitude indicating component 126 therein. The magnitude indicating component 126 is adapted to float on top of the non-compressible fluid and to move longitudinally within the magnitude indicating tube 124. The magnitude indicating tube 124 is made out of a substantially transparent or translucid material so as to allow visualization of the relative position of the magnitude indicating component 126 therein.

Typically, the non-compressible fluid is a suitable liquid while the magnitude indicating component 126 is a buoyant sphere. Also, typically, the magnitude indicating tube 124 is provided with magnitude indicating indicia (not shown) for allowing evaluation of the magnitude of the styptic pressure depending on the relative position of the magnitude indicating component 126 within the magnitude indicating tube 124.

Upon the styptic pressure being applied to the target anatomical site 12, the compressible bladder 122 is squeezed between the compressing surface 22 and the target anatomical site 12. Upon being squeezed, the compressible bladder 122 is compressed, hence forcing the non-compressible fluid into the magnitude indicating tube 124 and modifying the level of the magnitude indicating component 126 within the magnitude indicating tube 124.

In yet another alternative embodiment of the invention illustrated more specifically in FIGS. 16 through 18, the pressure sensor includes a pressure rod 128 mechanically coupled to the compressing surface, typically by the contacting tongue 48. The pressure rod 128 is slidably mounted within the hollowed-out compressing component 20 for longitudinal movement threrein towards the compressing component first longitudinal end 34 upon a pressure being exerted on the compressing surface.

The embodiment illustrated more specifically in FIGS. 16 through 18 also includes a biasing means (not shown) coupled to the pressure rod 128 for biasing the latter towards the compressing component second longitudinal end 16. A pressure indicator 130 is mounted on the compressing component 20.

A converting means 132 (shown schematically) is coupled to the pressure indicator 130 and to the pressure rod 128 for converting the movement of the pressure rod 128 into a pressure data indicated by the pressure indicator 130. The biasing means is typically calibrated so that the pressure data corresponds substantially to the magnitude of pressure exerted by the compressing surface 22 on the target anatomical area 12. Typically, although by no means exclusively, the pressure indicator 130 is a dial mounted on the grasping knob 34.

In use, as shown in FIG. 25, one of the initial steps for installing the styptic device 10 on the intended patient includes removing the peelable protective strip 94 from the double-sided adhesive tape 90.

As shown in FIG. 26, with the compressing component 20 in the stand-by configuration, the mounting component mounting plate 56 is then positioned using the first and second guiding indicia 106 and 108 so that compressing surface 22 is substantially in register with the target anatomical site 12. Digital pressure is applied on the top surface of the mounting component mounting plate 56 so as to adhesively secure the latter to the skin of the intended patient about the surrounding area 14.

The peelable protective strip 94 is then removed from the double-sided adhesive tape of the strap adhesive material 104 of the main strip section 102 and the first and second auxiliary strips 98, 100 for allowing the latter to be adhesively secured to the surrounding area 14. The main strip section 102 is wrapped around the thigh of the intended patient substantially from the groin area 88 of the latter to the first and second auxiliary strip sections 98, 100 so as to encircle the thigh of the intended patient such as shown in FIG. 27.

As illustrated more specifically in FIG. 28, the first and second auxiliary strip sections 98,100 are configured, sized and oriented relative to the mounting component mounting plate 56 so as to be located respectively substantially in register with the exterior face of the thigh and at least a portion the buttocks area of the intended patient. The sections of miniature hook and loop fibres allows the main strip section 102 to be releasably attached to the first and second auxiliary strip sections 98, 100 typically in a substantially transverse relationship relative to the latter.

This specific configuration is intended to optimize the distribution of pressure on the body of the patient so as to counteract the pressure exerted by the compressing component 20 with reduced risks of the base component 18 being lifted from the patient's body and with minimal discomfort to the patient.

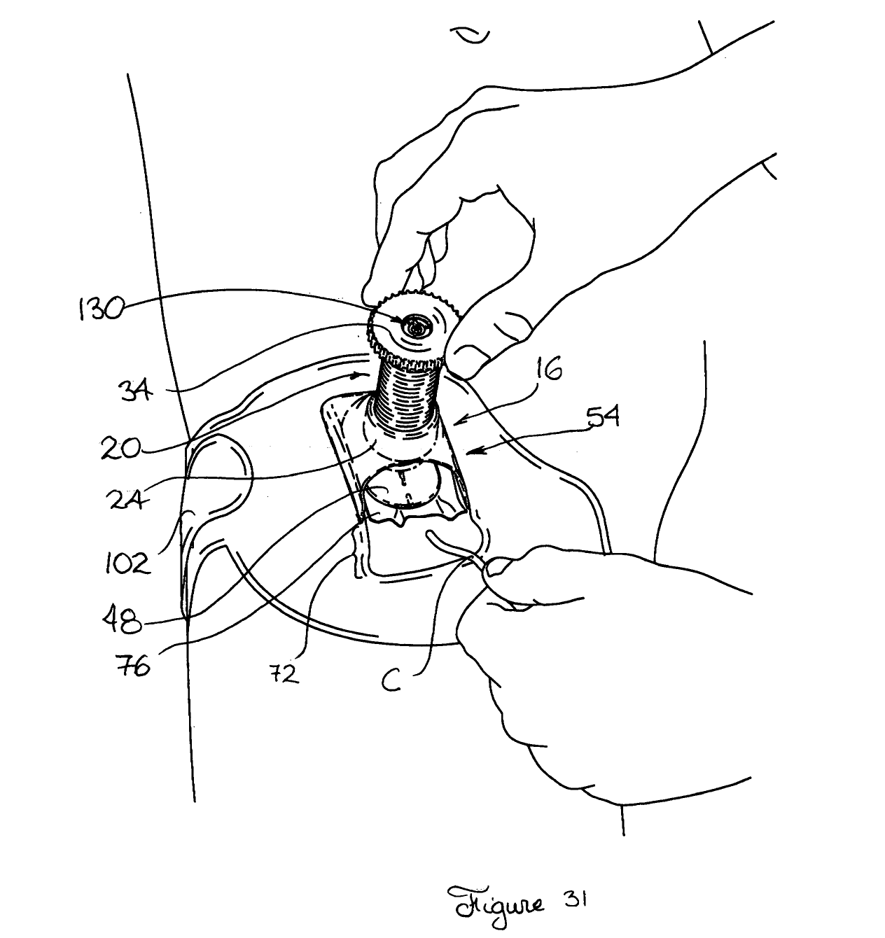

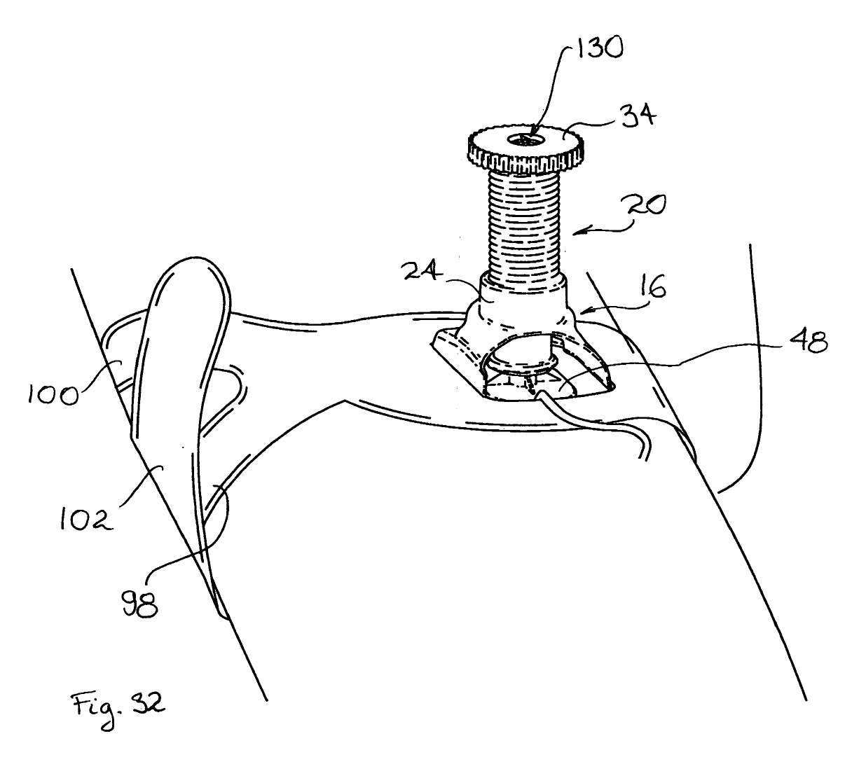

As illustrated in FIG. 29, a sterile gauze 76 is then typically inserted in the chamber 74 between the compressing surface 22 and the target anatomical site 12 through the peripheral wall access aperture 62.

As illustrated in FIG. 30, the grasping knob 42 is then grasped by the hand of an intended user to rotate the compressing component 20 so as to cause the lowermost thread of the second threaded portion 82 to disengage the coupling component internal thread 32 and allow the compressing component 20 to drop under the action of gravity towards the compressing component contacting configuration shown in FIG. 17.

As shown in FIG. 31, the other hand of the intended user may then be used to pull out the catheter C from the puncture site while the compressing component 20 is further rotated so as to cause the first threaded portion 80 to threadably engage the socket internal thread 32 for compressing the target anatomical site 12. Typically, rotation of the compressing component 20 while in the compressing configuration is continued until the exerted pressure reaches a predetermined threshold that can be monitored using the provided pressure sensor.

1. A styptic device for at least substantially hemostatically sealing a percutaneous vascular puncture formed in a body vessel and located at a target anatomical area of an intended patient, said target anatomical area being surrounded by a surrounding area of said intended patient, said styptic device comprising:
a base component positionable substantially adjacent said target anatomical site;
a mounting component coupled to said base component for mounting said base component on said intended patient in a substantially adjacent relationship relative to said target anatomical site;
a compressing component defining a compressing surface for exerting a compressing pressure on said target anatomical site, said compressing component being operatively coupled to said base component so as to be movable relative to the latter between a compressing configuration wherein said compressing surface exerts said compressing force on said target anatomical site and a retracted configuration wherein said compressing surface is spaced from said target anatomical site.

2. A styptic device as recited in claim 1 wherein
said base component includes a base component socket section, said base component socket section defining a socket channel extending therethrough, said socket channel being positionable substantially over said target anatomical site;
said compressing component extending at least partially through said socket channel for movement between said compressing and retracted configurations.

3. A styptic device as recited in claim 2 wherein
said compressing component includes a compressing component coupling section for coupling said compressing component to said base component, said compressing component coupling section having a substantially cylindrical configuration and being provided, at least in part, with a coupling component external thread;
said socket channel being provided, at least in part, with a socket internal thread for threaded engagement with said coupling component external thread.

4. A styptic device as recited in claim 3 wherein said compressing component defines a compressing component first longitudinal end and a longitudinally opposed compressing component second longitudinal end; said compressing component defining a compressing component grasping section located substantially adjacent said compressing component first longitudinal end for facilitating the manipulation of said compressing component and a compressing component contacting section for contacting said target anatomical site, said compressing component contacting section defining said compressing surface.

5. A styptic device as recited in claim 4 wherein said compressing component grasping section includes a grasping knob extending substantially radially outwardly from said compressing component adjacent said compressing component first longitudinal end.

6. A styptic device as recited in claim 4 wherein said compressing component contacting section includes a contacting tongue, said contacting tongue providing said compressing surface with a substantially rectangular configuration defining a contacting surface long axis and a substantially perpendicular contacting surface short axis, said contacting tongue being configured, sized and positioned so that said contacting surface long axis extends substantially in the direction of said body vessel when said compressing component is in said compressing configuration.

7. A styptic device as recited in claim 6 wherein said contacting tongue is releasably attached to said compressing component coupling section.

8. A styptic device as recited in claim 4 wherein said base component also includes a base component spacing section for maintaining said base component socket section in a substantially overlying spaced relationship relative to said target anatomical site.

9. A styptic device as recited in claim 8 wherein said mounting component includes a mounting component mounting plate extending from said base component for contacting at least part of said surrounding area, said mounting component mounting plate being provided with a mounting plate access aperture extending therethrough for allowing access to said target anatomical site; and wherein said base component spacing section includes a spacing section peripheral wall extending between said mounting component mounting plate and said base component socket section for maintaining said base component socket section in a spaced relationship relative to said mounting component mounting plate; said spacing section peripheral wall being provided with a peripheral wall access aperture extending therethrough for allowing access to said mounting plate access aperture.

10. A styptic device as recited in claim 9 wherein said spacing section peripheral wall includes a peripheral wall first section extending substantially perpendicularly from said mounting component mounting plate and a substantially frusto-conical peripheral wall second section extending from said peripheral wall first section, said peripheral wall second section defining a finger abutment area for allowing abutting contact therewith of some of the fingers of said intended user.

11. A styptic device as recited in claim 10 wherein said peripheral wall first and second sections are circumferentially truncated so as to define said peripheral wall access aperture, said peripheral wall access aperture defining a peripheral wall access aperture peripheral edge, said peripheral wall access aperture peripheral edge defining a pair of peripheral edge first segments formed in said peripheral wall first section and extending substantially perpendicularly from said mounting component mounting plate and a radially inwardly recessed peripheral edge second segment formed in said peripheral wall second section and extending between said peripheral edge first sections.

12. A styptic device as recited in claim 11 wherein said mounting plate access aperture defines a mounting plate access aperture protruding portion projecting outwardly from said spacing section beyond said peripheral wall access aperture peripheral edge.

13. A styptic device as recited in claim 9 wherein said spacing section defines a barrier receiving chamber for receiving a pressure transmitting contamination barrier component, said barrier receiving chamber being configured, positioned and sized so that said pressure transmitting contamination barrier component is positioned intermediate said compressing surface and said target anatomical site when said compressing component is in said compressing configuration.

14. A styptic device as recited in claim 13 wherein said peripheral wall first section is provided with barrier component receiving aperture extending therethrough for allowing at least a portion of said contamination barrier component to extend therethrough.

15. A styptic device as recited in claim 10 wherein said compressing component coupling section defines a first threaded portion located substantially towards said compressing component first longitudinal end, a second threaded portion located substantially towards said compressing component second longitudinal end and an unthreaded portion located intermediate said first and second threaded portions; the dimensional relationship between said base component socket section, said compressing component and said base component spacing section being such that
when said second threaded portion threadably engages said socket internal thread, said compressing component is in a stand-by configuration wherein said compressing component contacting section is located substantially adjacent said base component socket section above the level of said mounting component mounting plate so as to be in an overlying spaced apart relationship relative to said target anatomical site when said styptic device is mounted on said intended patient;
when the lowermost thread of said second threaded portion threadably disengages said coupling component internal thread so as to be moved underneath said socket channel, said unthreaded portion is put in register with said socket channel allowing said compression component to drop under the action of gravity to a compressing component contacting configuration wherein said lowermost thread of said first threaded portion is put into contact with said uppermost thread of said socket channel and said compressing surface is positioned substantially in register with said mounting component mounting plate so as to contact said target anatomical site when said styptic device is mounted on said patient;
when said first threaded portion threadably engages said coupling component internal thread, said compressing component is in said compressing configuration with said compressing surface located underneath said mounting component mounting plate so as to compress said target anatomical site when said styptic device is mounted on said patient.

16. A styptic device as recited in claim 9 wherein said mounting component mounting plate defines a mounting plate outer peripheral edge, said mounting plate outer peripheral edge being provided with a groin accommodating recess formed therein for accommodating the groin region of said intended patient.

17. A styptic device as recited in claim 9 wherein said mounting component also includes a mounting plate adhesive material attached to said mounting component mounting plate for adhesively securing said mounting plate to said surrounding area.

18. A styptic device as recited in claim 17 wherein said mounting plate adhesive material includes a double-sided adhesive tape, said double-sided adhesive tape having a first side thereof adhesively secured to the undersurface of said mounting component mounting plate and a second side thereof releasably covered by a peelable protective strip.

19. A styptic device as recited in claim 9 wherein said mounting component also includes at least one strap extending from said mounting component mounting plate for at least partially surrounding a body part of said intended patient.

20. A styptic device as recited in claim 19 wherein said mounting component includes a main strip section extending substantially laterally from said mounting component mounting plate and an auxiliary strip section extending from said mounting component mounting plate substantially opposite said main strip section said main and auxiliary strip sections being configured and sized for together encircling a body part of said patient and being provided with complementary main-to-auxiliary strap attachment means for releasable attachment threrbetween when together encircling said body part.

21. A styptic device as recited in claim 20 wherein said mounting component includes a first auxiliary strip section and a second auxiliary strip section both extending substantially laterally from said mounting component mounting plate, said first and second auxiliary strips diverging outwardly away from each other; said mounting component also including a main strip section extending from said mounting component mounting plate substantially opposite said first and second auxiliary strip sections; said first and second auxiliary strip sections and said main strip section being configured, sized and oriented so that said main strip section is adapted to substantially encircle said body part and releasably attach to said first and second auxiliary strip sections substantially transversally relative to the latter.

22. A styptic device as recited in claim 21 wherein said first and second auxiliary strip sections are configured, sized and oriented so as to extend substantially in register respectively with the external portion of the corresponding thigh and the buttocks region of said patient while said main strip section is adapted to extend from the corresponding groin region of said patient, substantially around the corresponding thigh of said patient to said first and second strip sections when said compressive surface is substantially in register with the femoral artery of said patient

23. A styptic device as recited in claim 21 wherein said main strip section and said first and second auxiliary strip sections are provided with portions of complementary miniature hook and loop fiber located adjacent distal ends thereof for allowing said main strip section to releasably attach to said first and second auxiliary strip sections.

24. A styptic device as recited in claim 9 wherein either one of said main strip section or said first and second auxiliary strip sections is provided with a strip adhesive material attached thereto for adhesively securing said either one of said main strip section or said first and second auxiliary strip sections to said surrounding area.

25. A styptic device as recited in claim 17 wherein said strip adhesive material includes a double-sided adhesive tape, said double-sided adhesive tape having a first side thereof adhesively secured to the undersurface of said either one of said main strip section or said first and second auxiliary strip sections and a second side thereof releasably covered by a peelable protective strip.

26. A styptic device as recited in claim 21 wherein said main strip section and said first and second auxiliary strip sections merge integrally into a strip connecting segment, said strip connecting segment being secured to said mounting component mounting plate.

27. A styptic device as recited in claim 26 wherein said base component is provided with at least one base component attachment flange extending substantially laterally from the lower peripheral edge of said peripheral wall first section, said at least one base component attachment flange being secured between said mounting component mounting plate and said strip connecting segment for securing said base component to said mounting component mounting plate.

28. A styptic device as recited in claim 1 further comprising an alignment means for facilitating the alignment of said pressure contacting surface with said body vessel.

29. A styptic device as recited in claim 28 wherein said alignment indicia includes a first alignment indicia located on said compressing component contacting section and a second alignment indicia located on said base component, said first and second alignment indicia extending in a substantially perpendicular relationship relative to each other and being located so that the intersection of the projection thereof defines an alignment intersection adapted to be put in register with said target anatomical area of said patient.

30. A styptic device as recited in claim 1 further comprising a pressure sensor for sensing the styptic pressure exerted by the compressing component on the target anatomical site and providing a usable indication of the magnitude of the styptic pressure.

31. A styptic device as recited in claim 30 wherein said pressure sensor provides an electrical output, said electrical output being transmittable through a corresponding sensor cable; whereby said sensor cable is adapted to be coupled to a pressure indicator.

32. A styptic device as recited in claim 30 wherein said pressure sensor includes a compressible bladder mounted on said compressing surface, said compressible bladder being fluidly coupled to a magnitude indicating tube, said compressible bladder being filled, at least in part, by a substantially non-compressible fluid, said magnitude indicating tube being provided with a buoyant magnitude indicating component adapted to float on top of said non-compressible fluid and to move longitudinally within said magnitude indicating tube; whereby upon said styptic pressure being applied to said target anatomical site, said compressible bladder will be squeezed between said compressing surface and said target anatomical site, upon being squeezed, said compressible bladder will be compressed, forcing said non-compressible fluid into said magnitude indicating tube and modifying the level of said magnitude indicating component within said magnitude indicating tube.

33. A styptic device as recited in claim 30 wherein said pressure sensor includes—a pressure rod mechanically coupled to said compressing surface, said pressure rod being slidably mounted within said compressing component for longitudinal movement threrein towards said compressing component first longitudinal end upon a pressure being exerted on said compressing surface;
a biasing means coupled to said pressure rod for biasing said compressing component second longitudinal end;
a pressure indicator mounted on said compressing component;
a converting means coupled to said pressure indicator and to said pressure rod for converting the movement of said pressure rod into a pressure data indicated by said pressure indicator; whereby said biasing means is calibrated so that said pressure data corresponds substantially to the magnitude of pressure exerted by said compressing surface on said target anatomical area.

34. A styptic device as recited in claim 33 wherein said a pressure indicator is a dial mounted on said grasping knob.
